(19) United States
(12) Patent Application Publication
(10) Pub. No.: US 2024/0216489 A1
(43) Pub. Date: Jul. 4, 2024

(54) IMMUNE TOLERANCE INDUCTION AND ERADICATION OF ANTI-DRUG ANTIBODIES (ADA) TO THERAPEUTIC FACTOR VIII

(71) Applicant: THE CHILDREN'S HOSPITAL OF PHILADELPHIA, Philadelphia, PA (US)

(72) Inventor: Valder R. Arruda, Philadelphia, PA (US)

(21) Appl. No.: 18/554,641

(22) PCT Filed: Apr. 20, 2022
(86) PCT No.: PCT/US22/25515

§ 371 Date: Oct. 10, 2023

Related U.S. Application Data
(60) Provisional application No. 63/177,650, filed on Apr. 21, 2021.

(51) Int. Cl. A61K 39/00; A61K 38/37; A61P 37/06

(52) U.S. Cl. CPC A61K 39/001; A61K 38/37; A61P 37/06; A61K 2039/5256

(57) ABSTRACT

Methods for immune tolerance induction and eradication of anti-drug antibodies to therapeutic Factor VIII are disclosed.

This application claims priority under 35 U.S.C. § 119(e) to U.S. Provisional Patent Application No. 63/177,650, filed Apr. 21, 2021. The foregoing application is incorporated by reference herein.

### FIELD OF THE INVENTION

The present invention relates to the fields of medicine and hematology. More specifically, the invention provides methods for immune tolerance induction and eradication of anti-drug antibodies (ADA) to therapeutic Factor VIII.

### BACKGROUND OF THE INVENTION

Several publications and patent documents are cited throughout the specification in order to describe the state of the art to which this invention pertains. Each of these citations is incorporated herein by reference as though set forth in full.

Hemophilia A (HA) is an X-linked bleeding disorder affecting ˜1/5,000 male births (Iorio, et al. (2019) Ann. Intern. Med., 171:540-546). Pathological variants in the FVIII gene lead to a deficiency of coagulation factor VIII (FVIII) activity. Patients with severe HA, defined as FVIII activity <1% of normal, have frequent spontaneous bleeds that can be life threatening. The bleeding phenotype is generally related to the residual factor activity: people with severe disease (factor activity <1% normal) have frequent spontaneous bleeds; people with moderate disease (factor activity 1%-5% normal) rarely have spontaneous bleeds, but bleed with minor trauma; and people with mild disease (factor activity 5%-40% normal) bleed during invasive procedures or trauma.

Intravenous (IV) FVIII concentrates can prevent and treat bleeding. However, 30% of severe HA and 10% of non-severe HA patients develop neutralizing alloantibodies, termed inhibitors, against FVIII (Hay, et al. (2011) Blood, 117(23):6367-70). Inhibitors are measured in Bethesda units (BU), where 1 BU neutralizes 50% FVIII activity. At high-titers (>5 BU), inhibitors render FVIII replacement therapy completely ineffective (Kempton, et al. (2014) Blood, 124(23):3365-72). Inhibitor formation mostly occurs within 20 FVIII exposures and is associated with a significant increase in both morbidity and mortality (Gouw, et al. (2007) Blood, 109(11):4648-54; Donfield, et al. (2007) Blood 110(10):3656-61; Witmer, et al. (2011) Br. J. Haematol., 152(2):211-6; Hoots, et al. (2008) Semin. Hematol., 45(2 Suppl 1):S42-9).

Management of inhibitor patients focuses on (a) hemostatic therapy with bypassing agents and (b) inhibitor eradication by immune tolerance induction (ITI) regimens. Bypassing agents, including activated prothrombin complex concentrate, activated factor VII (FVIIa), and emicizumab, circumvent the need of FVIII for hemostasis, but are costly and substantially inferior to FVIII in terms of procoagulant activity (Kempton, et al. (2014) Blood, 124(23):3365-72; Ferriere, et al. (2020) Blood 136(6): 740-748; Leissinger, et al. (2011) N. Engl. J. Med., 365(18):1684-92). Emicizumab, a bispecific antibody that partially mimics FVIII function, has recently been approved as a prophylactic therapy in HA patients (Ferriere, et al. (2020) Blood 136(6): 740-748; Mahlangu, (2018) N. Eng. J. Med., 379(9):811-822; Oldenburg, et al. (2017) N. Eng. J. Med., 377(9):809-818). However, patients continue to need hemostatic therapy while on emicizumab for break-through bleeding or surgical procedures. There are also serious safety concerns including thromboembolic complications and death using emicizumab in inhibitor patients (Oldenburg, et al. (2017) N. Engl. J. Med., 377(9):809-818). As such, inhibitor eradication remains a major therapeutic goal (Carcao, et al. (2019) Haemophilia, 25(4):676-684; Young, G. (2018) Blood Adv., 2(20):2780-2782; Escuriola-Ettingshausen, et al. (2020) Haemophilia DOI: 10.1111/hae.14010).

Immune tolerance induction (ITI) involves the frequent intravenous (IV) administration of high FVIII doses over a course of months to years with the goal of eradicating the inhibitor and the restoring the ability to use FVIII for hemostasis. However, ITI is only successful in 60-70% of patients with good prognostic features, as defined without any of the following risk factors: 1) an inhibitor titer greater than 10 BU prior to ITI start; 2) a historical titer greater than 200 BU; 3) a peak anamnestic titer after start of ITI of greater than 100 BU; 4) delay of greater than 1 year between diagnosis of ADA and initiation of ITI; and/or 5) a high risk FVIII genotype such as an inversion or large deletion. Even when successful, the burden of ITI includes high economic costs ˜$1 million/year per pediatric patient, the demands of frequent IV administrations, and thrombotic and infectious risks from central venous catheters (Van Dijk, et al. (2004) Haematologica 89(2):189-94). These burdens make ITI prohibitive for most patients outside the developed world and highlight the need for better regimens (Colowick, et al. (2000) Blood 96(5):1698-702).

### SUMMARY OF THE INVENTION

In accordance with the present invention, methods for inhibiting aberrant bleeding in a subject are provided, particularly in a subject with a defective Factor VIII (FVIII) gene. Methods for treating or inhibiting hemophilia A in a subject are also provided. Further, methods of reducing or eliminating FVIII inhibitors (e.g., neutralizing alloantibodies or anti-drug antibodies (ADA) to Factor VIII) in a subject are also provided. The methods comprise administering a nucleic acid molecule encoding Factor VIII (FVIII) to the subject. In certain embodiments, the subject has FVIII inhibitors of at least 10 Bethesda units (BU) prior to treatment. In certain embodiments, the subject has FVIII activity of less than 1% of normal prior to treatment. The methods of the instant invention may further comprise measuring the FVIII inhibitors in the subject prior to treatment. In certain embodiments, the nucleic acid molecule is contained within an expression vector such as a plasmid or viral vector (e.g., AAV). In certain embodiments, the nucleic acid molecule is administered to the liver of the subject. In certain embodiments, the methods of the instant invention result in the eradication of the FVIII inhibitors (e.g., a titer of less than 1 BU). In certain embodiments, the methods of the instant invention result in the subject having Factor VIII activity above 1%. In certain embodiments, the FVIII of the invention is FVIII ΔF+V3.

In accordance with another aspect of the instant invention, FVIII variants and nucleic acid molecules encoding the same are also provided.

### BRIEF DESCRIPTION OF THE DRAWINGS

FIG. 1A provides a graph of T regulatory cells (total pool of CD4+CD25+FoxP3+) in HA dogs with inhibitor (solid lines) and naïve dogs (dashed lines). FIG. 1B provides a graph of a representative of a HA dog with ADA by functional assay (Bethesda Unit, BU; circle), anti-canine IgG 2 (X), and canine FVIII B cells (diamond) post gene therapy at day zero.

FIG. 2 provides a graph of the expression of cFVIII variants in HA mice after AAV gene transfer.

FIGS. 3A and 3B provides graphs of high-responding inhibitor models T01 and T02, respectively, treated with AAV ITI, which resulted in inhibitor eradication. Arrow indicate treated bleeds. Dashed line defines negative inhibitor titer (<1 BU). T01 eradicated ADA by day 100 (Bethesda Unit <1%) and showed decline of anti-canine FVIII IgG2. Following immune tolerance, endogenous canine FVIII increased, reaching therapeutic levels of mild HA. T02 showed an anamnestic response characterized by an increase in ADA titers following gene therapy (peak 180 BU). However, the eradication and immune tolerance induction as well as endogenous expression of canine FVIII were all achieved with a delay when compared to T01 (no anamnestic response). FIG. 3C provides a graph of high-responding inhibitor models T03, which was not treated with AAV ITI.

FIG. 4 depicts the large FVIII gene deletion in a novel high-risk inhibitor model. The large gene deletion of FVIII increase the formation of ADA to >80% of patients with poor risk prognosis. The dog showed an anamnestic response characterized by an increase in ADA titers following gene therapy (peak 800 BU) followed by a significant decline in BU, anti-canine IgG2 ADA. Due to clinical issues the animal was sacrificed before complete immune tolerance induction.

FIG. 5 provides an amino acid sequence of FVIII (SEQ ID NO: 1). The amino acids at positions 560, 561, 659, 712, and/or 713 are bolded and underlined. The B domain is also indicated with italics and bolding. The provided amino acid sequence lacks the 19 amino acid signal peptide at the N-terminus (MQIELSTCFFLCLLRFCFS (SEQ ID NO: 2)).

### DETAILED DESCRIPTION OF THE INVENTION

Inhibitor patients can be classified as high or low responders. High responders have inhibitor titers that are high and/or develop high titers after FVIII exposure or even in the absence of additional FVIII exposure. In contrast, low responders have low inhibitor titers despite multiple FVIII challenges or only have transiently low titers. As low responders can be treated by high doses of FVIII, these patients face comparatively fewer complications than high responder who require bypassing agents for hemostasis. Risk factors for inhibitor formation include both environmental and genetic factors (DiMichele, D. M. (2013) Pediatr. Blood Cancer, 60 Suppl 1:S30-3). The genetic risk is best characterized by the underlying FVIII mutation with genotypes resulting in no FVIII cross-reactive material (CRM), CRM-negative, posing a higher risk for inhibitor development than CRM-positive genotypes. High-risk inhibitor mutations include large deletions, early nonsense mutations, and inversions (Oldenburg, et al. (2006) Haemophilia 12 Suppl 6:15-22), including F8 intron 22 inversion (F8-INV22) which is the most common FVIII genotype in severe HA (45%). Importantly, these mutations are also predictors of ITI failure (Coppola, et al. (2009) J. Thromb. Haemost., 7(11):1809-15). The F8-INV22 genotype also leads to severe CRM-negative HA in canine models.

Recently, multiple liver-directed AAV-based products in early-phase clinical trials for HA are reporting therapeutic FVIII levels (Peyvandi, et al. (2019) Haemophilia 25(5): 738-746; Perrin, et al. (2019) Blood 133(5):407-414; Gollomp, et al. (2019) Transfus Apher. Sci., 58(5):602-612). These AAV drugs differ in their vector serotype, FVIII transgene, and manufacturing process. Because the full-length FVIII exceeds the packaging capacity of AAV, all current approaches rely on B-domain deleted (BDD) FVIII variants (Samelson-Jones, et al. (2019) Mol. Ther. Methods Clin. Dev., 12:184-201), either the standard FVIII-SQ (Lind, et al. (1995) Eur. J. Biochem., 232:19-27) or an engineered BDD variant (e.g., FVIII-V3) that is associated with increased FVIII expression (McIntosh, et al. (2013) Blood 121(17):3335-44). Significantly, all trials excluded patients not previously heavily exposed to FVIII or patients with a history of inhibitors. Surprisingly, it is shown herein that AAV-mediated sustained expression of FVIII mimics ITI leading to successful eradication of FVIII inhibitors.

In accordance with the instant invention, it is desirable to greatly reduce, eliminate, or abolish pre-existing immune response to FVIII that is associated with both increased morbidity and mortality. Specifically, it is desirable to eradicated pre-existing allo-antibodies (ADA) against FVIII enzyme replacement therapy (ERT). It is demonstrated herein that this can be performed by (1) inducing and/or generating a subset of endogenous T cell that is only upregulated in patients with ADA and not among those patients without ADA; (2) induction of immune tolerance to ERT which will allows lifetime response to ERT; (3) in the case of the cDNA resulting in therapeutic levels of the missing protein, after immune tolerance induction (ITI), the continuous, uninterrupted endogenous production of FVIII will ameliorate the disease phenotype (uncontrolled or aberrant bleeding or hemophilia). More specifically, the methods of the invention may comprise (1) expression of a nucleic acid molecule encoding FVIII will not only eradicate pre-existing antibodies but also induce immune tolerance which will restore the ability of the patient to respond to FVIII; (2) generate endogenous T cells such as T regulatory cells and/or to down regulate pathological B cells to counter act the existing immune responses triggered by FVIII; and/or (3) delivered of the nucleic acid molecule by a viral vector (e.g., AAV) or a non-viral vectors (e.g., liposomes, naked cDNA, transposon) under the control of tissue-specific, non-tissue specific or ubiquitous elements or promoters).

Refractory patients are the most challenging to the eradication of ADA. This group is often described as those with: (a) unfavorable underlying mutation in the disease causative gene without cross reactive material (CRM negative, e.g., no circulating antigen detected by ELISA), (b) high titers of ADA prior to ITI, historical peak of ADA at any given time prior to ITI or anamnestic response after initiation of ITI; (c) prolonged time between the diagnosis of ADA and initiation of ITI; (d) children above 6 years of age, and/or (e) adults who failed to respond to the standard ITI. Poor prognostic factors in hemophilia with inhibitor include at least one factor: patients of >6 years of age, ITI started >1 year from inhibitor development, inhibitor peaks >200 BU, inhibitor titer >10 BU when ITI is started, and/or previously failed to at least of attempt of ITI.

Herein, it has been surprisingly shown that canine FVIII cDNA mediated ITI can successfully eradicate inhibitors in high-responding inhibitor HA dog models (e.g., >100 BU). These models recapitulate the most challenging patients that would likely benefit from endogenous upregulation of T reg cells in target species (canine, human) with high titers of ADA, but not in those without ADA following cDNA-mediated ITI. In this context, this invention provides (1) efficient eradication of high responding inhibitors, (2) long-term maintenance of FVIII immune tolerance, and (3) continuous FVIII expression that improves the bleeding phenotype after inhibitor eradication.

In accordance with the instant invention, methods for reducing, inhibiting, and/or blocking aberrant bleeding in a subject are provided. Methods for reducing and/or eliminating FVIII inhibitors in a subject are also provided. Additionally, methods for reducing and/or treating hemophilia A in a subject are provided. The method comprises administering a therapeutically effective amount of a nucleic acid molecule encoding FVIII to the subject. In a particular embodiment, the method comprises a single administration of the nucleic acid encoding FVIII to the subject. The methods may further comprise administering an additional agent which treats aberrant bleeding and/or hemophilia A. In a particular embodiment, the method further comprises administering at least one agent which induces and/or generates T reg cells (e.g., ATG). The additional agents may be administered at the same time (e.g., simultaneously) or sequentially (e.g., before or after) with the FVIII nucleic acid.

In certain embodiments, the subject has hemophilia A. The subject may have severe HA. In certain embodiments, the subject is a high responder. For example, the subject may have one or more high-risk inhibitor mutations within FVIII such as large deletions, early nonsense mutations, and inversions (e.g., F8 intron 22 inversion (F8-INV22)) (Oldenburg, et al. (2006) Haemophilia 12 Suppl 6:15-22). In certain embodiments, the subject has an inhibitor titer greater than 5 BU, greater than 10 BU, greater than 15 BU, greater than 20 BU, greater than 25 BU, greater than 30 BU, greater than 40 BU, greater than 50 BU, greater than 75 BU, or greater than 100 BU prior to treatment (e.g., without FVIII challenge). In certain embodiments, the subject has an inhibitor titer greater than 50 BU, greater than 75 BU, greater than 100 BU after a FVIII challenge. In certain embodiments, the subject has FVIII activity less than 1% of normal prior to treatment.

In certain embodiments, the treatment results in immune tolerance and/or eradication of the inhibitors (e.g., over time (e.g., within a year)). In certain embodiments, the inhibitor titer is less than 2 BU, particularly less than 1 BU, after treatment. In certain embodiments, the treatment results in an increase in FVIII activity to greater than 1% of normal, particularly greater than 2% or greater than 5%.

In accordance with the instant invention, novel Factor VIII variants are provided. The instant invention encompasses FVIII variants including FVIIIa variants and FVIII prepeptide variants. For simplicity, the variants are generally described throughout the application in the context of FVIII. However, the invention contemplates and encompasses Factor FVIIIa and FVIII prepeptide molecules as well as Factor VIII domain(s) (e.g., A1 and/or A2 domain) having the same amino acid substitutions and/or linkers as described in FVIII. In a particular embodiment, the FVIII variants of the instant invention are expressed as a single chain molecule or at least almost exclusively as a single chain molecule. In a particular embodiment, the FVIII variants are B-domain deleted (BDD) FVIII (optionally comprising a linker in place of the B-domain). In a particular embodiment, the FVIII variants comprise a light chain and a heavy chain (e.g., as a single chain molecule).

Full-length FVIII is a large, 280-kDa protein primarily expressed in liver sinusoidal endothelial cells (LSECs), as well as extra-hepatic endothelial cells (Fahs, et al., Blood (2014) 123:3706-3713; Everett, et al., Blood (2014) 123:3697-3705). FVIII predominantly circulates as a heterodimer of a heavy chain and a light chain bound through noncovalent metal-dependent interactions (Lenting, et al., Blood (1998) 92:3983-3996). Factor VIII comprises several domains. Generally, the domains are referred to as A1-A2-B-A3-C1-C2. The heavy chain of FVIII comprises A1-A2-B and the light chain comprises A3-C1-C2. Initially, FVIII is in an inactive form bound to von Willebrand factor (vWF). FVIII is activated by cleavage by thrombin (Factor IIa) and release of the B domain. The activated form of FVIII (FVIIIa) separates from vWF and interacts with coagulation factor Factor IXa—leading to the formation of a blood clot via a coagulation cascade.

The B domain comprises 40% of the protein (908 amino acids) and is not required for the protein procoagulant activity (Brinkhous, et al., Proc. Natl. Acad. Sci. (1985) 82:8752-8756). Notably, full-length FVIII cDNA (7 kb) exceeds the packing capacity of AAV vectors (˜4.7 kb). The removal of the B-domain of FVIII decreases the cDNA to ˜4.4 kb. AAV-based clinical trials for HA have reported positive results using this approach (Rangarajan, et al., N. Engl. J. Med. (2017) 377:2519-2530).

The most common B-domain deleted (BDD) FVIII comprises 14 original amino acid residues (SFSQNPPVLKRHQR (SEQ ID NO: 6)) as a linker (Lind, et al. (1995) Eur. J. Biochem., 232(1): 19-27). This BDD FVIII is typically referred to as BDD-SQ or hFVIII-SQ. This BDD FVIII form is commonly used to produce recombinant BDD-FVIII (˜ 4.4 Kb) as well for gene therapy (Berntorp, E., Semin. Hematol. (2001) 38(2 Suppl 4): 1-3; Gouw, et al., N. Engl. J. Med. (2013) 368:231-239; Xi, et al., J. Thromb. Haemost. (2013) 11:1655-1662; Recht, et al., Haemophilia (2009) 15:869-880; Sabatino, et al., Mol. Ther. (2011) 19:442-449; Scallan, et al., Blood (2003) 102:2031-2037). As noted above, gene therapy using AAV vectors can only use shortened FVIII molecules such as a BDD-FVIII due to the limited packaging capacity of the AAV (4.7 Kb) and other vector systems (Lind, et al. (1995) Eur. J. Biochem., 232(1):19-27). Short peptide linkers (e.g., 25 or fewer amino acids, 20 or fewer amino acids, 15 or fewer amino acids, or 10 or fewer amino acids) substituted for the B-domain can be used in FVIII variants (Lind, et al. (1995) Eur. J. Biochem., 232(1): 19-27; Pittman, et al., Blood (1993) 81:2925-2935; Toole, et al., Proc. Natl. Acad. Sci. (1986) 83:5939-5942, each incorporated by reference herein). U.S. Pat. No. 8,816,054, Pittman, et al. (Blood (1993) 81:2925-2935), and Toole, et al. (Proc. Natl. Acad. Sci. (1986) 83:5939-5942) (each incorporated by reference herein) also provides BDD FVIII molecules with linkers of different lengths and sequences. WO 2020/086686 (incorporated by reference herein) also provides BDD FVIII molecules with linkers of different lengths and sequences which have a reduced or minimized number of neo-epitopes in the linker region, thereby reducing adverse immunogenicity of the FVIII. In a particular embodiment, the linker comprises SFSQNPPVSK (SEQ ID NO: 3). This linker, for example, yields a furin-evading or resistant variant (ΔF) (e.g., resistant to cleavage by furin less than wild-type (e.g., B domain)).

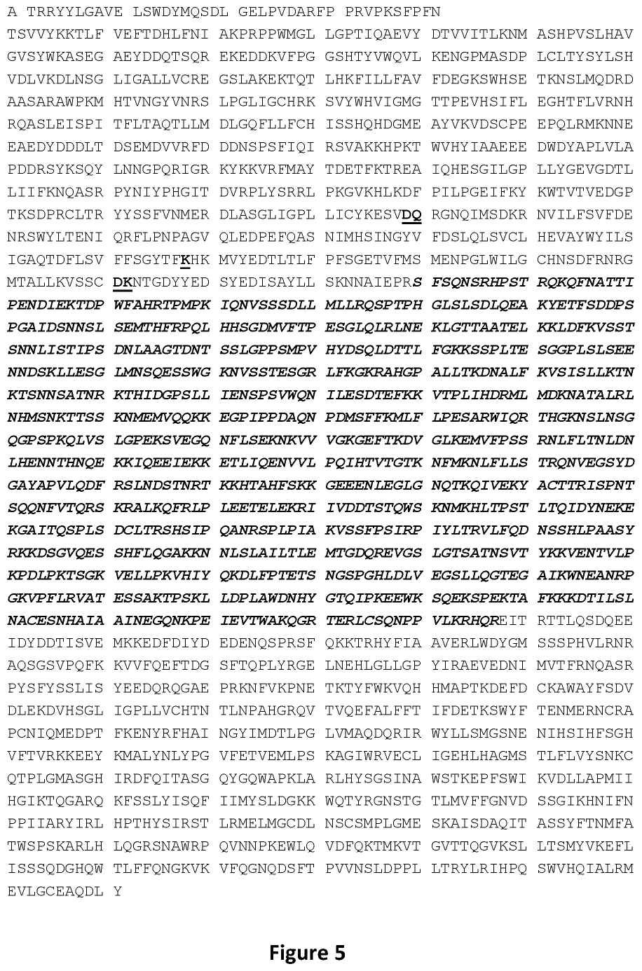

The FVIII of the instant invention can be from any mammalian species (e.g., human or canine). In a particular embodiment, the FVIII is human. Gene ID: 2157 and GenBank Accession Nos. NM_000132.3 and NP_000123.1 provide examples of the amino acid and nucleotide sequences of wild-type human FVIII (particularly the prepeptide comprising the signal peptide). FIG. 5 provides SEQ ID NO: 1, which is an example of the amino acid sequence of human FVIII. SEQ ID NO: 1 lacks the 19 amino acid signal peptide at its N-terminus (MQIELSTCFFLCLLRFCFS (SEQ ID NO: 2)). Nucleic acid molecules which encode Factor FVIII can be readily determined from the provided amino acid sequences as well as the provided GenBank Accession Nos.

The FVIII of the instant invention can be wild-type (not mutated) or be a variant (e.g., FVIII variants with increased activity). WO 2020/086686 (incorporated by reference herein) provides FVIII variants (e.g., which comprise at least one mutation at position 560, 561, 712, 713, and/or 659) which possess higher specific activity than wild type FVIII. In a particular embodiment, the FVIII of the instant invention has at least 75%, 80%, 85%, 90%, 95%, 97%, 99%, or 100% identity with SEQ ID NO: 1 (or an activated FVIII fragment thereof), particularly at least 90%, 95%, 97%, 99%, or 100% homology (identity).

In certain embodiments, the Factor VIII variant comprises a mutation at position 659. In a particular embodiment, the Lys (K) at position 659 is not substituted with a Pro (P), Gly (G), Met (M), or Leu (L). In a particular embodiment, the Lys at position 659 is substituted with Trp (W), Arg (R), Ala (A), His (H), Tyr (Y), Asp (D), Thr (T), Ser (S), Val (V), Phe (F), Gln (Q), or Cys (C). In a particular embodiment, the Lys at position 659 is substituted with Asp (D), Thr (T), Ser (S), Val (V), Phe (F), Gln (Q), or Cys (C). In a particular embodiment, the Lys at position 659 is substituted with Ser (S), Val (V), Phe (F), Gln (Q), or Cys (C). In a particular embodiment, the Lys at position 659 is substituted with Ser (S), Gln (Q), or Cys (C). In a particular embodiment, the Lys at position 659 is substituted with Gln (Q) or Cys (C).

In certain embodiments, the Factor VIII variant comprises a mutation at position 560. In a particular embodiment, the Asp (D) at position 560 is substituted with Ala (A), Val (V), Ile (I), Leu (L), His (H), Arg (R), or Lys (K). In a particular embodiment, the Asp (D) at position 560 is substituted with Ala (A), Val (V), Ile (I), or Leu (L). In a particular embodiment, the Asp (D) at position 560 is substituted with His (H), Arg (R), or Lys (K). In a particular embodiment, the Asp (D) at position 560 is substituted with Ile (I) or His (H).

In certain embodiments, the Factor VIII variant comprises a mutation at position 561. In a particular embodiment, the Gln (Q) at position 561 is not substituted with Leu (L), Arg (R), or Asn (N). In a particular embodiment, the Gln (Q) at position 561 is substituted with Asp (D) or Glu (E). In a particular embodiment, the Gln (Q) at position 561 is substituted with Asp (D).

In certain embodiments, the Factor VIII variant comprises a mutation at position 712. In a particular embodiment, the Asp (D) at position 712 is substituted with an amino acid other than Glu (E). In a particular embodiment, the Asp (D) at position 712 is substituted with Ala (A), Val (V), Ile (I), or Leu (L). In a particular embodiment, the Asp (D) at position 712 is substituted with Ile (I) or Leu (L). In a particular embodiment, the Asp (D) at position 712 is substituted with Leu (L).

In certain embodiments, the Factor VIII variant comprises a mutation at position 713. In a particular embodiment, the Lys (K) at position 713 is substituted with Ala (A), Arg (R), Met (M), Tyr (Y), Asp (D), Glu (E), Cys (C), or Gly (G). In a particular embodiment, the Lys (K) at position 713 is substituted with Arg (R), Met (M), Tyr (Y), Asp (D), Cys (C), or Gly (G). In a particular embodiment, the Lys (K) at position 713 is substituted with Asp (D) or Glu (E). In a particular embodiment, the Lys (K) at position 713 is substituted with Cys (C). In a particular embodiment, the Lys (K) at position 713 is substituted with Ala (A) or Gly (G). In a particular embodiment, the Lys (K) at position 713 is substituted with Gly (G).

The FVIII of the instant invention may have enhanced glycosylation (e.g., the V3 variant (McIntosh, et al. (2013) Blood, 121(17): 3335-44)). For example, the linker replacing the B domain comprises N-linked glycosylation triplets (e.g., 3-9, 4-8, 5-7, or 6 triplets (N-X-T/S)). In a particular embodiment, the FVIII comprises NATNVSNNSNTSNDSNVS (SEQ ID NO: 4), particularly in the linker (e.g., within the linker (e.g., within SEQ ID NO: 3) or adjacent to the linker replacing the B domain). In a particular embodiment, N-linked glycosylation triplets or SEQ ID NO: 4 is inserted between Q and N of SEQ ID NO: 3 in the linker replacing the B domain. In a particular embodiment, the linker replacing the B domain comprises SFSQNATNVSNNSNTSNDSNVSNPPVSK (SEQ ID NO: 5).

In a particular embodiment, the FVIII of the instant invention comprises the furin-evading linker and the enhanced glycosylation. As described hereinbelow, this combination leads to an improved effect on FVIII levels after AAV gene therapy.

Nucleic acid molecules encoding the FVIII may be prepared by any method known in the art. FVIII encoding nucleic acid molecules of the invention include cDNA, genomic DNA, RNA, and fragments thereof which may be single- or double-stranded. For example, nucleic acid molecules encoding the FVIII of the invention may be prepared by using recombinant DNA technology methods. The availability of nucleotide sequence information enables preparation of isolated nucleic acid molecules of the invention by a variety of means. For example, nucleic acid sequences encoding a FVIII may be isolated from appropriate biological sources using standard protocols well known in the art. As another example, two distinct rAAV production platforms are generally used for HA clinical trial vectors: 1) transfection of mammalian HEK293 cells with plasmid DNA (rAAV-293) or 2) transduction by recombinant baculoviruses into insect Sf9 cell lines (rAAV-Sf9) (Smith, et al. (2018) Cell Gene Ther. Insights 4:815-825; Rumachik, et al. (2020) Molecular Therapy: Methods Clin. Development, 18:P98-118; Li, et al. (2020) Nat. Rev. Genet., 21(4):255-272; Kondratov, et al. (2017) Molecular Therapy 25(12):2661-2675).

The nucleic acid molecules may be maintained in any convenient vector, particularly an expression vector. Nucleic acids of the present invention may also be maintained as RNA or DNA in any convenient vector or cloning vector. In a particular embodiment, the nucleic acids may be maintained in a vector suitable for expression in mammalian cells, particularly human cells. Vectors such as those described above comprise the regulatory elements necessary for expression of the DNA in the host cell positioned in such a manner as to permit expression of the DNA in the host or target cell (e.g., hepatocyte). Such regulatory elements required for expression include, but are not limited to, promoter sequences, transcription initiation sequences, and enhancer sequences.

Generally, the nucleic acid molecules of the instant invention will be administered to a subject in a composition comprising at least one carrier. Except insofar as any conventional carrier is incompatible with the nucleic acid to be administered, its use in the pharmaceutical composition is contemplated. In a particular embodiment, the carrier is a pharmaceutically acceptable carrier for injection. In certain embodiments, the nucleic acid molecules of the instant invention are administered to a subject in a cell (e.g., a hepatocyte) wherein the cell may be maintained in a composition comprising at least one carrier.

As explained herein, FVIII nucleic acids of the instant invention may be used, for example, as therapeutic and/or prophylactic agents which modulate the blood coagulation cascade, particularly in subjects with defective, deleted and/or inactive FVIII (e.g., subjects with hemophilia A). It is demonstrated herein that the FVIII nucleic acid molecules provide effective hemostasis.

In a particular embodiment of the present invention, FVIII nucleic acid molecules may be administered to a patient via injection in a biologically or pharmaceutically compatible carrier, e.g., via injection into the liver. The FVIII nucleic acid molecules of the invention may optionally be encapsulated into liposomes or mixed with other phospholipids or micelles. FVIII nucleic acid molecules may be administered alone or in combination with other agents known to modulate hemostasis. An appropriate composition in which to deliver the FVIII nucleic acid molecules may be determined by a medical practitioner upon consideration of a variety of physiological variables, including, but not limited to, the patient's condition and hemodynamic state. A variety of compositions well suited for different applications and routes of administration are well known in the art and are described hereinbelow.

The preparation containing the FVIII nucleic acid molecules may contain a physiologically acceptable matrix and is formulated as a pharmaceutical preparation. The preparation can be formulated using substantially known prior art methods, it can be mixed with a buffer containing salts, such as NaCl, CaCl₂), and amino acids, such as glycine and/or lysine, and in a pH range from 6 to 8. Until needed, the purified preparation containing the FVIII nucleic acid molecules can be stored in the form of a finished solution or in lyophilized or deep-frozen form. In a particular embodiment, the preparation is stored in lyophilized form and is dissolved into a visually clear solution using an appropriate reconstitution solution. Alternatively, the preparation according to the present invention can also be made available as a liquid preparation or as a liquid that is deep-frozen. The preparation according to the present invention may be especially stable, i.e., it can be allowed to stand in dissolved form for a prolonged time prior to application.

The preparation according to the present invention can be made available as a pharmaceutical preparation with the FVIII nucleic acid molecules in the form of a one-component preparation or in combination with other factors in the form of a multi-component preparation.

FVIII encoding nucleic acids may be used for a variety of purposes in accordance with the present invention. In a particular embodiment of the invention, a nucleic acid delivery vehicle (e.g., an expression vector such as a viral vector or plasmid) for modulating blood coagulation or treating hemophilia A is provided wherein the expression vector comprises a nucleic acid sequence coding for FVIII as described herein. Administration of the FVIII encoding expression vectors to a patient results in the expression of the FVIII which serves to alter the coagulation cascade and/or eliminate inhibitors. In accordance with the present invention, a FVIII encoding nucleic acid sequence may encode a variant polypeptide as described herein whose expression increases hemostasis. In a particular embodiment, the nucleic acid sequence encodes a human FVIII.

Expression vectors comprising FVIII nucleic acid sequences may be administered alone, or in combination with other molecules useful for modulating hemostasis. According to the present invention, the expression vectors or combination of therapeutic agents may be administered to the patient alone or in a pharmaceutically acceptable or biologically compatible composition.

In a particular embodiment of the invention, the expression vector comprising nucleic acid sequences encoding the FVIII is a viral vector. Viral vectors which may be used in the present invention include, but are not limited to, adenoviral vectors (with or without tissue specific promoters/enhancers), adeno-associated virus (AAV) vectors of multiple serotypes (e.g., AAV-1 to AAV-12, particularly AAV-2, AAV-5, AAV-7, and AAV-8) and hybrid AAV vectors, lentivirus vectors and pseudo-typed lentivirus vectors (e.g., Ebola virus, vesicular stomatitis virus (VSV), and feline immunodeficiency virus (FIV)), herpes simplex virus vectors, vaccinia virus vectors, and retroviral vectors. In a particular embodiment, the vector is an adeno-associated virus (AAV) vector. In a particular embodiment, the vector is a lentiviral vector.

In a particular embodiment of the present invention, methods are provided for the administration of a viral vector comprising nucleic acid sequences encoding a FVIII. Adeno-associated virus vectors of utility in the methods of the present invention may include at least the essential parts of adeno-associated virus vector DNA. As described herein, expression of a FVIII following administration of such an adeno-associated virus vector serves to modulate hemostasis, particularly to enhance the procoagulation activity.

Recombinant adeno-associated virus vectors have found broad utility for a variety of gene therapy applications. Their utility for such applications is due largely to the high efficiency of in vivo gene transfer achieved in a variety of organ contexts.

Adeno-associated virus particles may be used to advantage as vehicles for adequate gene delivery. Such virions possess a number of desirable features for such applications, including: structural features related to being a double stranded DNA nonenveloped virus and biological features such as a tropism for the human respiratory system and gastrointestinal tract. Moreover, adeno-associated viruses are known to infect a wide variety of cell types in vivo and in vitro by receptor-mediated endocytosis.

For some applications, an expression construct may further comprise regulatory elements which serve to drive expression in a particular cell (e.g., hepatocyte) or tissue type (e.g., liver). Such regulatory elements are known to those of skill in the art. The incorporation of tissue specific regulatory elements in the expression constructs of the present invention provides for at least partial tissue tropism for the expression of the FVIII. For example, hematopoietic or liver specific promoters may also be used.

As explained hereinabove, AAV for recombinant gene expression have been produced in the human embryonic kidney cell line 293 (Wright, Hum Gene Ther (2009) 20:698-706; Graham et al. (1977) J. Gen. Virol. 36:59-72). Briefly, AAV vectors are typically engineered from wild-type AAV, a single-stranded DNA virus that is non-pathogenic. The parent virus is non-pathogenic, the vectors have a broad host range, and they can infect both dividing and non-dividing cells. The vector is typically engineered from the virus by deleting the rep and cap genes and replacing these with the transgene of interest under the control of a specific promoter. For recombinant AAV preparation, the upper size limit of the sequence that can be inserted between the two ITRs is about 4.7 kb. Plasmids expressing a FVIII under the control of the CMV promoter/enhancer and a second plasmid supplying adenovirus helper functions along with a third plasmid containing the AAV-2 rep and cap genes may be used to produce AAV-2 vectors, while a plasmid containing either AAV-1, AAV-6, or AAV-8 cap genes and AAV-2 rep gene and ITR's may be used to produce the respective alternate serotype vectors (e.g., Gao et al. (2002) Proc. Natl. Acad. Sci. USA 99:11854-11859; Xiao et al., (1999) J. Virol. 73:3994-4003; Arruda et al., (2004) Blood 103:85-92). AAV vectors may be purified by repeated CsCl density gradient centrifugation and the titer of purified vectors determined by quantitative dot-blot hybridization.

From the foregoing discussion, it can be seen that FVIII expressing nucleic acid vectors may be used in the treatment of disorders associated with aberrant blood coagulation, particularly hemophilia A.

The expression vectors of the present invention may be incorporated into pharmaceutical compositions that may be delivered to a subject, so as to allow production of a biologically active protein (e.g., a FVIII) or by inducing expression of the FVIII in vivo by gene- and or cell-based therapies or by ex vivo modification/transduction of the patient's or donor's cells. In a particular embodiment of the present invention, pharmaceutical compositions comprising sufficient genetic material to enable a recipient to produce a therapeutically effective amount of a FVIII can influence hemostasis in the subject. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents (e.g., co-factors) which influence hemostasis.

In particular embodiments, the pharmaceutical compositions also contain a pharmaceutically acceptable excipient/carrier. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of FVIII, such labeling could include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques and guidance provided in the present invention. Therapeutic doses will depend on, among other factors, the age and general condition of the subject, the severity of the aberrant blood coagulation phenotype, and the strength of the control sequences regulating the expression levels of the polypeptide. Thus, a therapeutically effective amount in humans will fall in a relatively broad range that may be determined by a medical practitioner based on the response of an individual patient to vector-based treatment.

FVIII encoding vectors of the present invention may be administered to a patient by any means known. Direct delivery of the pharmaceutical compositions in vivo may generally be accomplished via injection using a conventional syringe. In this regard, the compositions may be delivered subcutaneously, epidermally, intradermally, intrathecally, intraorbitally, intramucosally, intraperitoneally, intravenously, intraarterially, orally, intrahepatically or intramuscularly. In a particular embodiment, the FVIII encoding nucleic acid molecules are administered by injection. In a particular embodiment, the FVIII encoding nucleic acid molecules are administered to the bloodstream. In a particular embodiment, the FVIII encoding nucleic acid molecules are administered to the liver.

### Definitions

The following definitions are provided to facilitate an understanding of the present invention.

The singular forms “a,” “an,” and “the” include plural referents unless the context clearly dictates otherwise.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote. With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

With respect to protein, the term “isolated protein” is sometimes used herein. This term may refer to a protein produced by expression of an isolated nucleic acid molecule of the invention. Alternatively, this term may refer to a protein which has been sufficiently separated from other proteins with which it would naturally be associated (e.g., so as to exist in “substantially pure” form). “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “vector” refers to a carrier nucleic acid molecule (e.g., RNA or DNA) into which a nucleic acid sequence can be inserted for introduction into a host cell where it will be replicated. An “expression vector” is a specialized vector that contains a gene or nucleic acid sequence with the necessary regulatory regions (e.g., promoter) needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.), particularly at least 75% by weight, or at least 90-99% or more by weight of the compound of interest. Purity may be measured by methods appropriate for the compound of interest (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N. Y.; and Kibbe, et al., Eds., Handbook of Pharmaceutical Excipients, American Pharmaceutical Association, Washington.

As used herein, the term “subject” refers to an animal, particularly a mammal, particularly a human.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., aberrant bleeding) resulting in a decrease in the probability that the subject will develop the condition.

A “therapeutically effective amount” of a compound or a pharmaceutical composition refers to an amount effective to prevent, inhibit, treat, and/or lessen the symptoms of a particular disorder or disease.

The following example is provided to illustrate various embodiments of the present invention. The example is illustrative and is not intended to limit the invention in any way.

### EXAMPLE

Canine HA models are the ideal preclinical experimental system to test hemophilia therapies. They are outbred, long-lived, naturally occurring, large animal models that recapitulate the HA bleeding phenotype. HA dogs are the only HA animal model that consistently develops inhibitors in a species-specific manner and their immune response against canine (c) FVIII targets the analogous epitopes as the patient immune response against human (h) FVIII (Ozelo, et al. (2014) Blood 123(26):4045-53; Nguyen, et al. (2014) Blood 123(17):2732-9). Like the patient anti-FVIII immune response, canine neutralizing antibodies are also closely associated with a single IgG subtype (IgG2 for dogs). There are 2 established HA dog models, with both models sharing the same high-risk for inhibitor F8-INV22 genotype (Hough, et al. (2002) Thromb. Haemost., 87(4):659-65; Lozier, et al. (2002) Proc. Natl. Acad. Sci., 99(20): 12991-6). However, they are different breeds with different genetic backgrounds. As discussed hereinbelow, new high-responding inhibitor models are described including a large F8 deletion that likely represents the worst-case clinical scenario. HA dogs also have spontaneous bleeds, and, therefore, better capture the role of tissue damage and inflammation in the pathological immune response to FVIII (Kurnik, et al. (2010) Haemophilia, 16(2):256-62). Lastly, inhibitor dogs can be followed for years making them a unique model that allows for the FVIII immune response to be longitudinally studied over clinically relevant time frames (e.g., for >9 years).

In contrast, HA rodent models are of limited value for testing whether AAV-FVIII gene therapy can eradicate pre-existing inhibitors and induce immune tolerance. HA mice are an inbred model with a limited lifespan and with the strength of the immune response highly dependent on the background strain (Qadura, et al. (2011) Haemophilia 17(2):288-95). HA mice also only develop xenoimmune responses to hFVIII, but generally not alloimmune responses to murine FVIII (Doering, et al. (2002) Thromb. Haemost., 88(3):450-8). Further, AAV-expressing human FVIII transgene injection at week 12 into HA mice failed to reduce inhibitor titer by week 23 as treated mice showed high titers of inhibitor by Bethesda assay (between 100-200 BU) and IgG to hFVIII.

Likewise, naïve wild-type nonhuman primates (NHPs) also develop a xenoprotein response against hFVIII after AAV gene therapy that overtime results in a severe bleeding phenotype due to cross reactivity between hFVIII and endogenous NHP-FVIII (McIntosh, et al. (2013) Blood 121(17):3335-44; Bunting et al. (2018) Mol. Ther., 26:496-509). Despite immunosuppression to lower the inhibitor titer and maintain the experiment, there was no evidence of immune tolerance for >20-weeks in these NHPs.

AAV serotype 8 (AAV8) cFVIII vectors (5×10¹³ vg/kg; FVIII-SQ) have been delivered to four HA dogs (UNC K01, UNC K03, UNC L44, and QU Wembley), each with F8-INV22 (Finn, et al. (2010) Blood 116(26):5842-8). All four HA dogs had a BU of 4 or lower at the start of therapy. In three of the dogs, treatment resulted in a transient increase in inhibitor titers up to 7 BU followed by therapeutic cFVIII levels (activity of 1.5%-8.0%) that decreased bleeding by 92%. In the fourth dog, a remarkable increase in inhibitor titers was seen after vector administration. The inhibitors showed a strong anamnestic response and peaked at a titer at 216 BU. While the titers slowly decreased over 80 weeks (but without eradication (e.g., <1 BU)), cFVIII activity levels remained below the limit of detection (<1%) and without elimination of the bleeding phenotype. Notably, in high-responder patients, ITI failure rates increase to more than double that of non-high-responders.

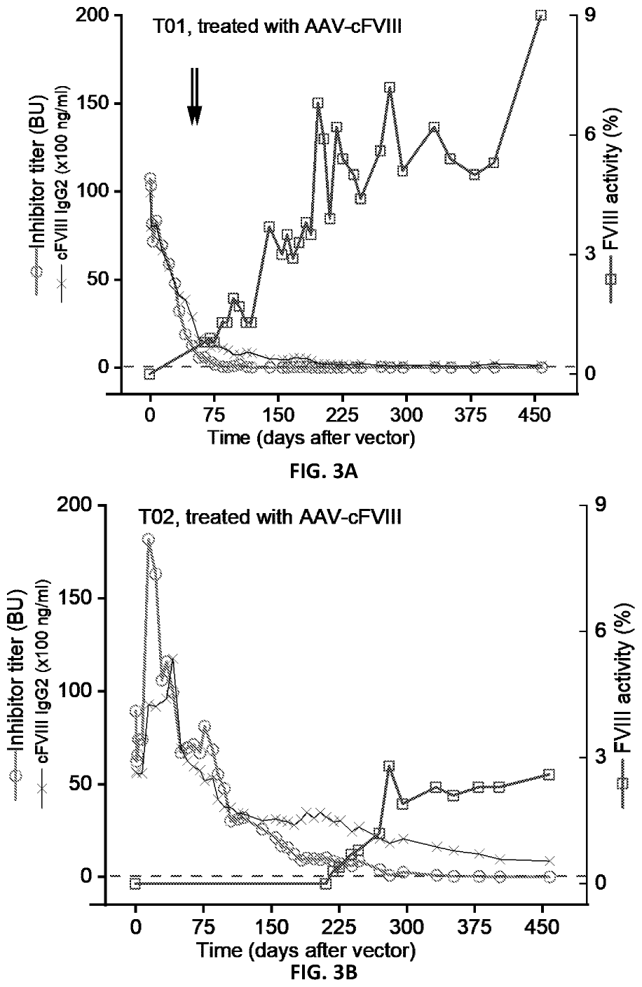

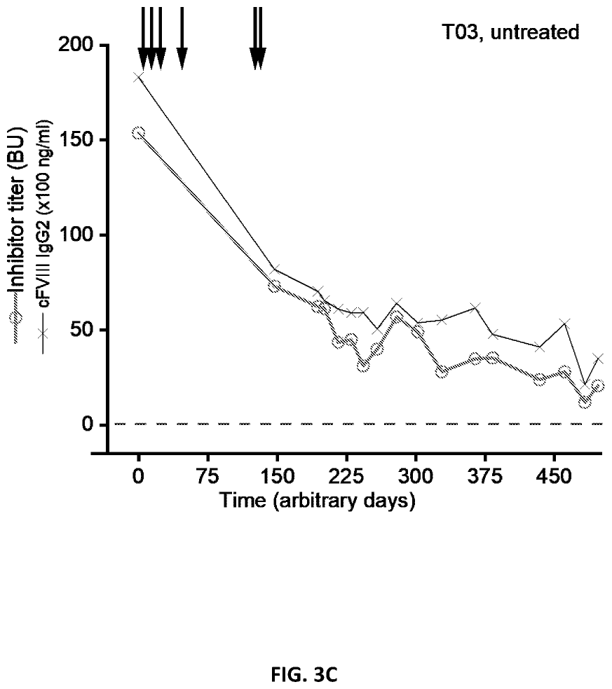

Spontaneous inhibitor eradication in canine HA inhibitor models has not been observed without AAV ITI. A UNC low responding dog (M78) consistently maintained his anamnestic response with low inhibitor titers for more than 700 days. M78 consistently showed anamnestic response of low intensity (less than 4 BU) upon exposure to canine FVIII protein. Persistent inhibitors were also observed in the absence of AAV ITI in 4 additional UNC dogs and in a high-responding Texas model HA (T03) (FIG. 3): 2,505 days of cumulative observation.

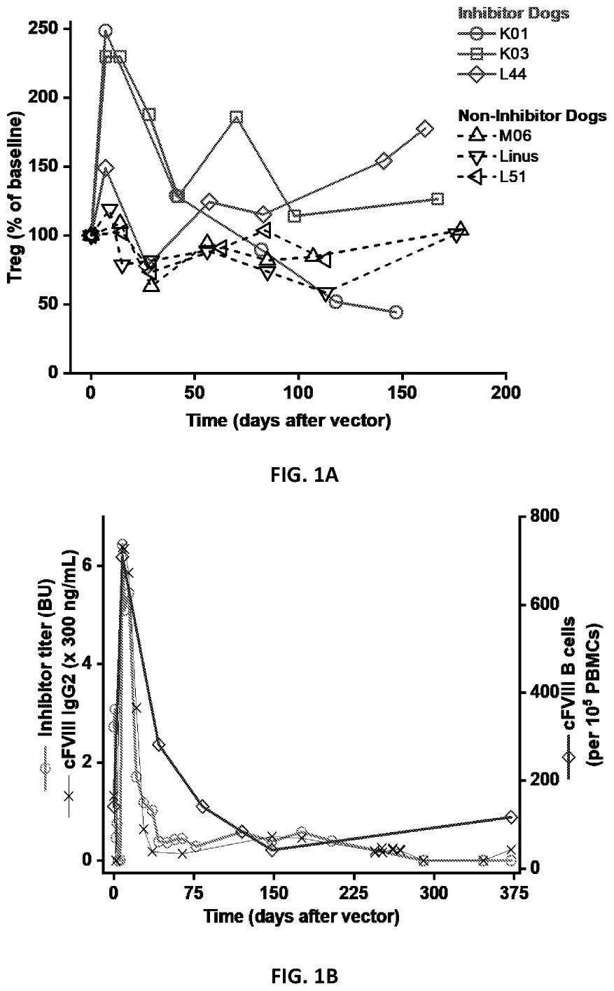

Notably, an early increase of the total T reg cell-population in the HA inhibitor dogs is observed <2 weeks after receiving AAV-cFVIII, but this T reg expansion was not seen in non-inhibitor HA dogs (FIG. 1A). This indicates that T reg cells have an early role in successful AAV ITI. Consistent with this hypothesis, intensive T cell directed immune suppression with antithymocyte globulin (ATG) concomitantly administered with AAV vector results in an increase of transgene immunogenicity in NHPs, while delaying ATG by 5 weeks does not (Baker, et al. (2010) Self Nonself 1(4):314-322). Thus, generation of T regs following AAV ITI is an early event and the perturbation of T reg cells at early time points around AAV delivery can be detrimental to transgene immune tolerance. FIG. 1B provides a graph of a representative of a HA dog with ADA by functional assay (Bethesda Unit, BU; circle), anti-canine IgG 2 (X), and canine FVIII B cells (diamond) post gene therapy at day zero.

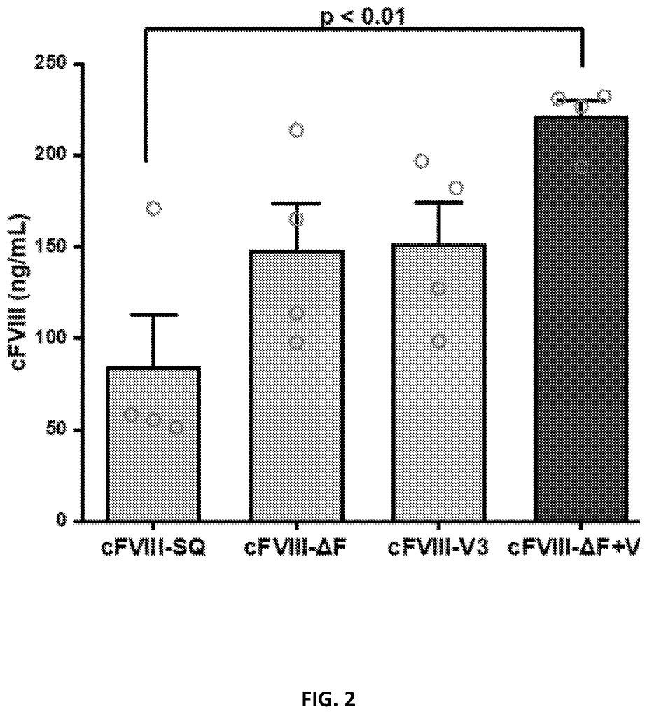

A novel BDD variant (cFVIII-ΔF+V3) is described herein that combines the advantages of a furin-evading variant (ΔF) (Siner, et al. (2016) JCI Insight, 1(16):e89371) with the enhanced glycosylation of the V3 variant (McIntosh, et al. (2013) Blood, 121(17): 3335-44). This combined approach has an improved effect on cFVIII levels after AAV gene therapy (FIG. 2).

Novel high-responding inhibitor dog models were fostered from three unrelated breeds. In one model (Texas), three animals have HA due to F8-INV22. Male littermates (T01, T02, and T03) all developed high-titer high-responding inhibitors after treatment with cFVIII concentrates for bleeding. Prior to treatment, T01 and T02 had high titers of 107 BU and 89 BU. T01 and T02 were treated with AAV-cFVIII gene therapy (9.0×10¹² vg/kg; FVIII-ΔF+V3) that resulted in unexpected inhibitor eradication and anti-cFVIII IgG2 normalization (FIGS. 3A and 3B). This inhibitor eradication was consistent with stringent immune tolerance induction ((1) inhibitor eradication (<1 BU) and no anti-FVIII IgG; (2) no recurrence of ADA upon subsequent cFVIII challenges; and (3) appropriate pharmacokinetics after recombinant cFVIII infusion) as demonstrated by challenges with cFVIII-SQ protein. These results indicate that the cFVIII-ΔF+V3 and cFVIII-SQ have similar immunogenicity consistent with the known immunogenicity of the FVIII B-domain (Samelson-Jones, et al. (2019) Mol. Ther. Methods Clin. Dev., 12: 184-201). After inhibitor eradication, cFVIII activity was measurable with levels between 2-7% normal without spontaneous bleeding episodes. T01 and T02 both tolerized within 1 year of gene therapy (16 weeks and 48 weeks, respectively), which is highly encouraging given the clinical experience of patients with poor prognostic features. T02's kinetics of ITI were longer than T01's due to an early anamnestic response.

T03 was not treated because of an unrelated neurological condition (FIG. 3C). For T03, both the inhibitor and anti-cFVIII IgG2 persisted with inhibitor titers remaining ˜50 BU, even in the absence of cFVIII protein infusions. This clinical course is consistent with a high-responding phenotype. The course of T03 augments the observations of the persistent low-responding inhibitor in an untreated HA dog. There is no evidence of spontaneous eradication of inhibitors. Combined, these data indicate that AAV ITI is effective in challenging high inhibitor models and that the anti-cFVIII immune response persists without AAV ITI.

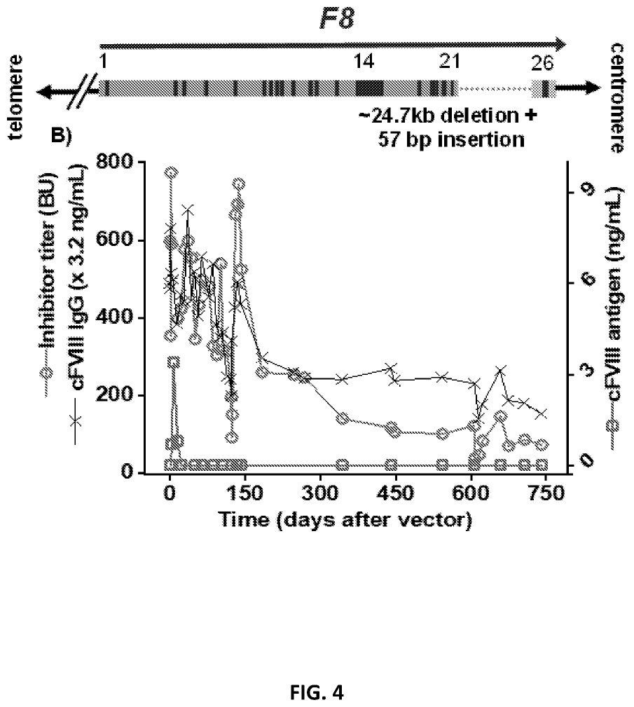

In another model (Ohio), the HA phenotype in this dog is due to a large cFVIII multi-exon deletion (exons 21-25) with a 57 bp inserted fragment of unknown origin (FIG. 4). In HA patients, large FVIII deletions are associated with high-risk of inhibitor development and unfavorable prognosis for ITI. This animal had inhibitor titers >500 BU prior to treatment, which is higher than any previous dog. Thus, his combination of a very high inhibitor titer and large FVIII gene deletion represents the worst-case clinical situation. This dog was treated with AAV-cFVIII (6.0×10¹² vg/kg; FVIII-ΔF) (FIG. 4). An initial spike of cFVIII antigen level was observed, as previously seen in other dogs one week after vector. His course was prolonged by an anamnestic response that occurred on day 121 after a major bleed with life-threatening anemia (hemoglobin 3 g/dl) requiring whole blood transfusion and bypassing therapies. Subsequently, he demonstrated steadily declining inhibiter titer and anti-cFVIII IgG2. However, he was euthanized for humane reasons after another life-threating hemorrhage. At the time of sacrifice, his inhibitor titer and declined >90% its peak value. Though of limited success due to the unexpected hemorrhage and the exceptionally high starting BU and large FVIII gene deletion, this model supports the conclusion that AAV gene therapy promotes cFVIII tolerance in high responders.

To date, 9 HA inhibitor dogs of 5 different breeds have been treated with AAV-cFVIII gene therapy and 6 additional untreated HA inhibitor dogs have been followed. Except for the extreme model of >500 BU, all AAV-cFVIII treated dogs eventually tolerized, while none of untreated animals tolerized. Survival analysis demonstrates a significant difference between the AAV-cFVIII treated and untreated animals. By 2 years, 80% of the treated dogs have been tolerized. This compares very favorably to the 35% of subjects in clinical ITI trials with good prognostic features that tolerized at 2 years (Hay, et al. (2012) Blood 119(6):1335-44).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for reducing or eliminating Factor VIII (FVIII) inhibitors and/or treating hemophilia A in a subject, said method comprising administering a nucleic acid molecule encoding FVIII to said subject,
wherein said subject has FVIII inhibitors of at least 10 Bethesda units (BU) prior to said treatment.

2. The method of claim 1, wherein said subject has FVIII inhibitors of at least 25 BU prior to said treatment.

3. The method of claim 1, further comprising the step of measuring the FVIII inhibitors in said subject prior to administration of the nucleic acid molecule.

4. The method of claim 1, wherein said nucleic acid molecule is contained within an expression vector.

5. The method of claim 4, wherein said expression vector is a plasmid.

6. The method of claim 4, wherein said expression vector is a viral vector.

7. The method of claim 6, wherein said viral vector is an adeno-associated virus vector.

8. The method of claim 1, wherein said nucleic acid molecule is administered to the liver of the subject.

9. The method of claim 1, wherein said administration results in eradication of the FVIII inhibitors.

10. The method of claim 1, wherein said administration results in the subject having less than 1 BU of Factor VIII inhibitor.

11. The method of claim 1, wherein the FVIII activity in said subject is less than 1%.

12. The method of claim 1, wherein said administration results in the Factor VIII activity in the subject increasing to above 1%.

13. The method of claim 1, wherein said administration results in the Factor VIII activity in the subject increasing to above 2%.

14. The method of claim 1, wherein the B domain is replaced with a linker that is furin resistant and comprises N-linked glycosylation triplets.

15. The method of claim 1, wherein said subject has FVIII inhibitors of at least 100 BU after challenge with FVIII.

16. The method of claim 1, wherein said subject has FVIII inhibitors of at least 100 BU prior to said treatment.

17. The method of claim 1, wherein said FVIII lacks a B domain.

18. The method of claim 17, wherein the B domain is replaced with an amino acid sequence comprising SEQ ID NO: 3 and N-linked glycosylation triplets, optionally wherein said N-linked glycosylation triplets are inserted into SEQ ID NO: 3.

19. The method of claim 18, wherein the B domain is replaced with an amino acid sequence comprising SEQ ID NO: 3 and SEQ ID NO: 4.

20. A Factor VIII (FVIII) variant wherein the B domain has been replaced with an amino acid sequence comprising SEQ ID NO: 3 and N-linked glycosylation triplets, optionally wherein said N-linked glycosylation triplets are inserted into SEQ ID NO: 3.

21. The FVIII variant of claim 20, wherein the B domain has been replaced with an amino acid sequence comprising SEQ ID NO: 3 and SEQ ID NO: 4.

22. A nucleic acid molecule encoding the FVIII variant of claim 20.


SEQUENCE LISTING (6 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 19 aa, Artificial Sequence
  Signal peptide
MQIELSTCFFLCLLRFCFS

SEQ ID NO: 3 - protein, 10 aa, Artificial Sequence
  Linker
SFSQNPPVSK

SEQ ID NO: 4 - protein, 18 aa, Artificial Sequence
  glycosylation sequence
NATNVSNNSNTSNDSNVS

SEQ ID NO: 5 - protein, 28 aa, Artificial Sequence
  B domain linker
SFSQNATNVSNNSNTSNDSNVSNPPVSK

SEQ ID NO: 6 - protein, 14 aa, Artificial Sequence
  BBD linker
SFSQNPPVLKRHQR